(19) United States
(12) Patent Application Publication
(10) Pub. No.: US 2025/0285346 A1
(43) Pub. Date: Sep. 11, 2025

(54) METHOD FOR ACQUIRING AND RECONSTRUCTING IMAGES OF A HEART IN FREE BREATHING, SYSTEM

(71) Applicants: UNIVERSITE DE BORDEAUX, BORDEAUX (FR); CENTRE HOSPITALIER UNIVERSITAIRE DE BORDEAUX, TALENCE cedex (FR); FONDATION BORDEAUX UNIVERSITÉ, BORDEAUX (FR); CENTRE HOSPITALIER UNIVERSITAIRE VAUDOIS, LAUSANNE (CH)

(72) Inventors: Matthias STUBER, ROMANEL-SUR-LAUSANNE (CH); Aurélien BUSTIN, BORDEAUX (FR); Hubert COCHET, BORDEAUX (FR)

(21) Appl. No.: 18/858,658

(22) PCT Filed: Apr. 20, 2023
(86) PCT No.: PCT/EP2023/060330

§ 371 Date: Oct. 21, 2024

(30) Foreign Application Priority Data
Apr. 22, 2022 (FR) FR2203782

(51) Int. Cl. G06T 11/60; A61B 5/00; A61B 5/055; A61B 5/366; G01R 33/56; G06T 7/33; G06T 7/38; G06T 11/00

(52) U.S. Cl. CPC G06T 11/60; A61B 5/0044; A61B 5/055; A61B 5/366; A61B 5/7292; A61B 5/742; G01R 33/5608; G06T 7/337; G06T 7/38; G06T 11/001; G06T 2207/10016; G06T 2207/10088; G06T 2207/30048; G06T 2210/41; G06T 2211/424

(57) ABSTRACT

A Method for reconstructing an image of a patient's heart from a magnetic resonance imaging device generating a magnetic field, includes acquisition of the electrical activity of the patient's heart; generation of an MRI sequence and acquisition of a 2D black blood image; generation of an MRI sequence and acquisition of a 2D bright blood image; application of a non-rigid registration algorithm to each subset of black blood images for each heart section; application of a non-rigid registration algorithm to each subset of bright blood images for each heart section; generation of a set of 2D images for each section plane, each image corresponding to a combination made between each pair of black blood and bright blood images.

### FIELD OF THE INVENTION

The invention relates to the field of magnetic resonance imaging, notably cardiac magnetic resonance imaging. It relates more particularly to late gadolinium enhancement (LGE) magnetic resonance imaging MRI. It applies to cardiac imaging, but also to angiography. The field of the invention more particularly pertains to imaging methods and devices for detecting and locating a scar in myocardial tissue.

### PRIOR ART

The reference technique for the assessment of regional scar formation and myocardial fibrosis is bright blood late gadolinium elevation or BR-LGE (for BRight-Blood LGE) imaging. In this type of imaging, the cancelation of the viable myocardial signal is brought about using inversion-recovery pulses, which allows scars to be visualized with a high contrast between healthy myocardial tissue and scars. However, for small scars or myocardial scars adjacent to the blood chambers of the heart, the high intensity of the signal from the blood prevents clear visualization and accurate delimitation of scars, in particular subendocardial scars. In order to circumvent this problem, BL-LGE (BLack-blood LGE) imaging techniques have been proposed. They make it possible to cancel out signals from healthy myocardium and blood simultaneously, thereby providing high contrast between both scars and blood and between scars and healthy myocardium.

The cancelation of the blood signal, to obtain a “black blood” contrast, is obtained by applying, to an area to be imaged of a patient, a radio frequency (RF) inversion-recovery sequence with a 180° pulse followed by a preparation module and a read module. The time between the preparation module that occurs immediately after the transmission of a magnetization inversion radiofrequency pulse and the read sequence is called inversion time TI.

The aim is to acquire the signals to generate the image of the area to be imaged in such a way as to locate and detect a scar present in the myocardial tissues.

A drawback of solutions of the prior art known as black blood-bright blood acquisition is that they require a significant acquisition time due to the 3D acquisition involving many calculations. The patient is under these conditions in free breathing. One consequence is the need to apply image correction algorithms requiring large computational resources involving computational times lengthening the acquisition sequence and a difficulty of applying them in real time. However, when the acquisition time is extended, the variability of the consequences of breathing leads to the production of image processing artifacts affecting the legibility of the latter. These artifacts accentuate the difficulty of reconstructing sharp and accurate images in order to locate and detect the scar. In addition, long MRI acquisitions are uncomfortable for the patient. A duration of 10 to 20 min is considered as a very long duration and it is difficult for the patient to stay within the MRI without making a movement.

Today, these sequences require an acquisition time of more than 15 min. The acquired images are unsatisfactory in numerous cases, because the artifacts from an image extracted from a section plane of the 3D image lead to cases where it is impossible to discriminate the presence of a potential scar from the presence of blood located near the muscle. Indeed, in some cases, the scar is so close to blood, it is known as subendocardial, that it is difficult to know whether it is a scar, blood or an image artifact. One problem is to base a clinical decision leading to a misdiagnosis on the presence of a scar or not.

Another solution consists in making a quick acquisition in apnea, but this solution results in the need to reduce the acquisition time from a few seconds to a few minutes and often involves having to reduce the area to be imaged, for example by limiting it to a 3D portion.

However, it is nevertheless necessary to obtain a minimum of images to reconstruct a 3D portion. One problem is that heartbeats also influence the measurement and involve making acquisitions synchronized on the heartbeat. This constraint imposes reducing the number of potential acquisitions over a given portion of time. Indeed, it is necessary to sequence the acquisitions on the heart rhythm, so it is necessary to spread these acquisitions over a minimum number of beats. Acquisitions in apnea greatly constrain the acquisition capacity.

Finally, there are 2D acquisition methods, but these do not make it possible to obtain sufficient resolution and contrast to detect and locate scars. It would be necessary to perform a plurality of 2D acquisitions of a same area to unambiguously identify certain cases, but variations in breathing and heartbeats over time do not allow 2D images to be taken on identical section planes of the organ over time.

### SUMMARY OF THE INVENTION AND ADVANTAGES

According to a first aspect, the invention relates to a method for reconstructing an image of a patient's heart from a magnetic resonance imaging device generating a magnetic field comprising:
A first phase of image generation including:
   Acquisition of the electrical activity of the patient's heart;
   Generation of a first 180° inversion radio frequency signal to switch a longitudinal magnetization of tissues of an imaged area, said first inversion radio frequency signal being generated between two QRS complexes of the acquired electrical activity, so-called first inter-beat phase;
   Generation of a first magnetization preparation including a set of pulses following the generation of the first radio frequency inversion signal in the same first inter-beat phase;
   Acquisition of a first 2D image by a magnetization measurement of the imaged area, said acquisition being performed after a first predefined duration following the generation of the first magnetization preparation, said acquisition being synchronized with the heart rhythm at a first time marker of the same inter-beat phase;
   Generation of a second magnetization preparation including a set of pulses in a second inter-beat phase following the first inter-beat phase;
   Acquisition of a second 2D image by a magnetization measurement of the imaged area, said acquisition being performed after a second predefined duration following the generation of the second magnetization preparation, said acquisition being synchronized with the acquisition of a heart rhythm at the same first time marker of the second inter-beat phase as the first marker of the first inter-beat phase;
Repetition of the first phase to generate a plurality of first images and a plurality of second images for a plurality of section planes of the heart in inter-beat phases following the first and second inter-beat phases, each pair of first image and second image being associated with a section plane;
   Generation of a set of third 2D images for each section plane, each third image corresponding to a first combination made between each pair of first merged images and second merged images for each section plane.

This aspect of the invention relates to the case where an image per section plane is acquired. This possibility is offered for fast acquisitions requiring thirty or so beats to cover fifteen or so section planes with one black blood image and one bright blood image per section. However, this sequence may be improved by acquisition in free breathing by acquiring a plurality of images per section plane for black blood on the one hand and for bright blood on the other hand.

According to another aspect, the invention relates to a method for reconstructing an image of a patient's heart from a magnetic resonance imaging device generating a magnetic field comprising:
A first phase of image generation including:
   Acquisition of the electrical activity of the patient's heart;
   Generation of a first 180° inversion radio frequency module so as to switch a longitudinal magnetization of an imaged area, said first inversion radio frequency signal being generated between two QRS complexes of the acquired electrical activity, so-called first inter-beat phase;
   Generation of a first magnetization preparation including a set of pulses following the generation of the first radio frequency inversion module in the same first inter-beat phase;
   Acquisition of a first 2D image by a magnetization measurement of the imaged area, said acquisition being performed after a first predefined duration following the generation of the first magnetization preparation, said acquisition being synchronized with the heart rhythm at a first time marker of the same inter-beat phase;
   Generation of a second magnetization preparation including a set of pulses in a second inter-beat phase following the first inter-beat phase;
   Acquisition of a second 2D image by a magnetization measurement of the imaged area, said acquisition being performed after a second predefined duration following the generation of the second magnetization preparation, said acquisition being synchronized with the acquisition of a heart rhythm at the same first time marker of the second inter-beat phase as the first marker of the first inter-beat phase;
Repetition of the first phase to generate a plurality of first images and a plurality of second images for a plurality of section planes of the heart in inter-beat phases following the first and second inter-beat phases, each pair of first image and second image being associated with a section plane;
Repetition of the first phase to generate a plurality of first images and second images of a same section plane of the heart in inter-beat phases following the first and second inter-beat phases.

Put another way, the method comprises the repetition of the first phase, to generate a subset of first images and a subset of second images of each section plane of a plurality of section planes of the heart, in inter-beat phases following the first and second inter-beat phases.

The method also comprises:
Application of a non-rigid registration algorithm to each subset of first images of a same section plane to register said first images together;
Application of a non-rigid registration algorithm to each subset of second images of a same section plane to register said second images together;
Merging, on the one hand, the first images registered together to produce a first merged image and, on the other hand, the second images registered together to produce a second merged image;
Generation of a set of third 2D images for each section plane, each third image corresponding to a first combination made between each pair of first merged images and second merged images for each section plane.

One advantage is to allow acquisitions in free breathing of short durations to be produced not requiring the computational times of a 3D acquisition.

Advantageously, the method comprises the following steps:
merging, for each of the section planes, on the one hand, the first images registered together to produce a first merged image and, on the other hand, the second images registered together to produce a second merged image;
generating, for said section planes, a set of third 2D images, each third image corresponding to a first combination made between the first merged image and the second merged image produced for one of the section planes.

Advantageously:
for each section plane, a non-rigid registration algorithm is applied to the first images of the section plane to register said first images of the subset of first images together;
for each section plane, a non-rigid registration algorithm is applied to the first images of the section plane to register said first images of the subset of first images together.

According to one embodiment, the first duration being determined such that the first images acquired are images having a first contrast making it possible to display black blood images and the second duration being determined such that the second images acquired are images having a second contrast making it possible to display bright blood images.

According to one embodiment, the registration step is followed by the steps of:
Averaging the first registered acquired images to produce a first merged image for each section plane;
Averaging the second registered acquired images to produce a first merged image for each section plane.

One advantage is to increase the signal to noise ratio, also called SNR.

According to one embodiment, the first duration is the inversion time in inversion-recovery.

Advantageously, the first duration is defined such that the longitudinal magnetization of blood and that of healthy myocardium are nulled at the same instant during the acquisition of the first 2D image.

According to one embodiment, the determination of the first duration is automatically calculated from a computer-implemented method of calculating an optimal inversion time performed from a processing of at least one image acquired from an MRI pre-sequence.

This calculation is for example performed by the method, implemented by computer, for determining the optimal inversion time described in patent application FR2203766. The MRI pre-sequence comprises the acquisition of a plurality of MRI images by acquisition sequences having distinct respective inversion times. The inversion time is calculated from these images.

According to one embodiment, the method comprises a step of coloring the pixels of each first merged image having a luminance greater than a given threshold, the coloring step being performed prior to the first combination. In the exemplary case, the colored images correspond to black blood images.

One advantage is to allow a scar near the myocardium and blood volume(s) to be located and identified safely, i.e. unambiguously.

According to one embodiment, the first combination is an overlay of the first image on the second merged image. That is to say an overlay of the colored black blood image on the colored bright blood image.

In one particular embodiment, the first image is the first merged image.

According to one embodiment, each new generation of a plurality of first images and second images of a same section plane is performed after each repetition of the first phase to generate a plurality of first images and a plurality of second images for a plurality of section planes of the heart.

According to one embodiment, each generation of a new first image and a new second image in a new section plane of the heart is performed after the set of repetitions of the first phase to generate a plurality of first images and second images of a previous section plane of the heart.

One advantage is to take all the images of a same section plane over a short time interval to avoid image shift drifts in a same section plane when they are interlaced together after the acquisition of different images of other section planes.

According to one embodiment, the method includes:
a display of a set of first merged images for each section plane;
a display of a set of second merged images for each section plane;
a display of a set of third 2D images for each section plane;
each first image of a given section plane being displayed in the immediate vicinity of the second image and the third image of the same given section plane.

Advantageously, the first image displayed and the second image displayed for a section plane are respectively the first merged image and the second merged image.

According to one embodiment, the first merged image for each section plane corresponds to a black blood image of a section of the heart, the second merged image for each section plane corresponds to a bright blood image of a section of the heart, the third image for each section plane corresponding to an image of a section of the organ on which a scar, if present, is displayed in color.

According to one embodiment, the first and second magnetization preparation is a T1rho weighting T1, said method being performed during late elevation after injection of gadolinium.

According to one embodiment, the first magnetization preparation is a preparation of T1rho type, said T1rho preparation being defined by the sequencing of a configuration of the magnet defined as follows: 90x-SLy-180y-SL-y-SLy-180-y-SL-y-90-x.

According to another exemplary preparation of the T1rho type, another magnetization configuration may be for example 90x-Sly-90-x.

According to one embodiment, the first magnetization preparation is a preparation of the T2prep type or a preparation of the MTC type.

According to one embodiment, the generation of a second magnetization preparation including a set of pulses in an inter-beat phase is followed by a step of filtering the acquired images corresponding to fatty areas of the imaged organ.

According to one embodiment, the first images and the second images are acquired successively in a synchronized manner with the electrical activity of the heart between two QRS complexes for a plurality of minutes in free breathing during a single examination.

According to one embodiment, the first images and the second images are acquired on a set of 8 to 20 section planes of the organ, the phase of reiteration of the acquisitions of a plurality of first images and second images in a same section plane making it possible to acquire between 2 and 10 images of first images and between 2 and 10 images of second images per section plane.

According to another aspect, the invention relates to a magnetic resonance imaging system comprising a magnetic field generator and a radio frequency device, an electrocardiogramd a processing device, a display for displaying the generated images, said system being configured to implement the method of the invention.

One advantage is to enable the generation of images in which it is possible for a radiologist to locate and detect the scar of myocardial tissues in a reliable manner.

One advantage is to make it possible to display an area of interest such as a scar in color.

Another advantage is to make it possible to acquire images in free breathing quickly, notably without requiring the patient to have the imaging performed in apnea.

Furthermore, the method according to the invention is simple, fast and inexpensive in terms of calculations. It is easily integrated into an existing MRI system without modification.

One advantage is that for each given section of the heart, a plurality of images are collected, which makes it possible to reduce noise, or increase the signal to noise ratio and reduce residual artifacts.

One advantage of using a non-rigid algorithm is to mitigate movement artifacts related to variation in breathing. The implementation of such an algorithm makes it possible to increase the signal to noise ratio.

Finally, 2D acquisition enables faster and more robust acquisition than 3D acquisition.

### BRIEF DESCRIPTION OF THE FIGURES

Other characteristics and advantages of the invention will become clearer upon reading the following detailed description, in reference to the appended figures, that illustrate:

FIG. 1: an exemplary embodiment of a system of the invention including an MRI device, an electrocardiogramalculation for performing the processing operations of the acquired images and a human-machine interface for viewing the images;

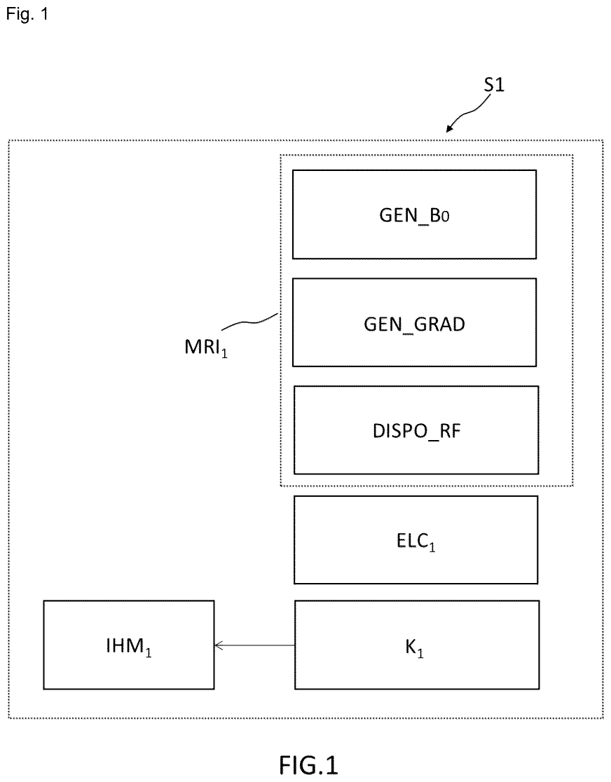

FIG. 2: a schematic representation of a portion of the sequence generated by an MRI device on two heart beats,

FIG. 3: a schematic representation of a black blood and bright blood MRI acquisition phase performed on a plurality of heart beats,

FIG. 4: a schematic representation of a 3D heart illustrating the different section planes and the images acquired in each section plane,

FIG. 5: a flowchart of the steps of the method according to one embodiment of the invention.

### DESCRIPTION OF THE INVENTION

### MRI System

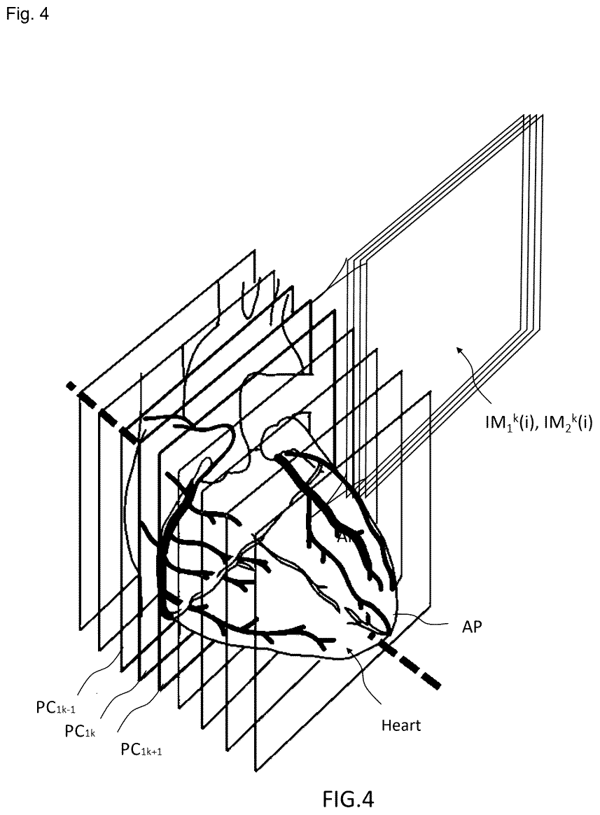

FIG. 4 schematically represents an imaging system S₁ including a magnetic resonance device MRI₁ according to the invention comprising a set of excitation and measurement equipment, a calculator K₁ to perform the processing on the acquired images and a display IHM₁ to display the generated images.

In a manner known per se, the set of excitation and measurement equipment comprises an MRI imaging device comprising a static magnetic field generator GEN_B₀ comprising a polarization main magnet, a gradient generator GEN_GRAD, and a radio frequency device DISPO_RF comprising a radio frequency signal generator and a radio frequency antenna.

The static magnetic field generator GEN_B₀ comprises a magnet intended to generate a substantially uniform static polarization magnetic field in a polarization area (generally a tunnel) intended to comprise the area to be imaged of the patient, for example the heart.

The gradient generator GEN_GRAD comprises three gradient coils or solenoids disposed and configured to vary the intensity of the magnetic field in the polarization area along respective orthogonal axes conventionally noted x, y, and z fixed with respect to the polarization area. The choice of the intensities circulating in these coils makes it possible to select, from several possible, a thickness and a section plane in which the magnetization of the area to be imaged of the patient received in the polarization area will be measured.

The radio frequency device GEN_RF comprises coils or solenoids and is capable of generating MRI acquisition sequences comprising magnetization preparation sequences of the area to be imaged and signal read sequences from the area to be imaged. Each acquisition sequence comprises at least one radio frequency pulse of predetermined and controllable frequency, shape, duration, phase, amplitude.

The preparation module, also called preparation or preparation phase, is configured to excite, i.e. change the direction of magnetization of the tissues of the area to be imaged, i.e. modulate their magnetization. It is noted TAprep or TBprep or instead T1rho in the remainder of the description.

The read module is configured to measure the magnetization of the area to be imaged resulting from the preparation sequence. The read module is also referred to as the acquisition module or read phase or read window.

In other words, the images are acquired by magnetization measurements of the area to be imaged.

The assembly advantageously comprises an electrocardiogra ELC₁ intended to acquire an electrocardiogram ECG₁ of the patient.

The imaging system comprises a processing device K₁ configured to perform calculations notably registration of images and averaging of said images. The processing device further makes it possible to color the images.

The imaging system S1 further comprises a display for displaying the two-dimensional images in grayscale and/or in color of the area to be imaged from the signals measured by the RF magnetic field generator and processed by the processing device.

According to one embodiment, the reconstructed image is a color and grayscale matrix image. It comprises a set of pixels each characterized by an intensity I able to take a set of M values (M being a finite integer greater than 1) corresponding to M gray levels ranging from 0 and M-1. For example, this value can take 256 values comprised between 0 and 255. This value may be higher in other embodiments. According to one embodiment, the images may be partially colored for a selection of pixels having a luminance greater than a given threshold.

Representations of the images generated by the processing device K₁ are intended to be displayed on a screen of the human-machine interface IHM₁ of the imaging system S₁.

### Acquisition Sequence

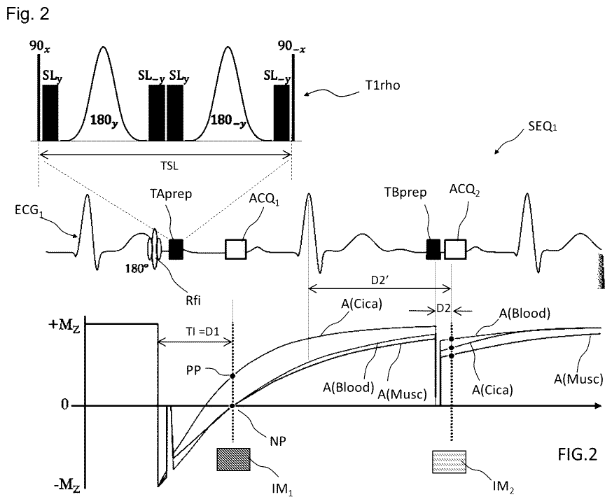

FIG. 2 shows an example of a portion of black blood and bright blood MRI acquisition sequence SEQ₁ of a myocardial image as well as the electrical activity ECG₁ represented on two consecutive beats. An electrocardiogram ECG₁ acquired by the electrocardiograph ECL₁during two heartbeats as well as the acquisition sequence SEQ₁ applied to the area to be imaged are overlaid in order to better represent the time-related characteristics of each of these curves. The ECG acquisition is a step noted ACQ₀. The lower part of FIG. 2 represents the variation of the longitudinal magnetization Mz of the tissues of the area to be imaged as a function of time t as well as two images IM₁ and IM₂ reconstructed from signals measured during the sequence.

Preferably, the invention makes it possible to synchronize the MRI excitation and acquisition sequences on the electrocardiogram ECG₁. This synchronization makes it possible on the one hand to take measurements at instants when the heart is most stable and on the other hand to take measurements at equivalent instants between two same positions of the heart in order to compare images of the same section. A way of synchronizing these excitations and acquisitions and triggering the measuring device on the QRS complex.

In the remainder of the description, the first image IM₁ will refer to a black blood acquired image and the second image IM₂ will refer to a bright blood acquired image. According to one example, throughout the sequence, from the first beat during which the image acquisitions are performed, the odd beats are used to acquire the black blood images and the even beats are used to acquire the bright blood images. This example will be described in more detail further on in the description. However, other configurations may be envisaged for executing the method of the invention, for example associating the odd beats with the acquisition of bright blood images and the even beats with black blood acquisition.

The invention also applies in the case where the bright blood images would be acquired before the black blood images. The invention also applies in the case where the acquired black blood and bright blood images would not be acquired immediately on two consecutive beats, but for example spaced by one or more beat(s) without measurement. However, a benefit of acquiring these images on two consecutive beats is to minimize the acquisition time and therefore to minimize the effects of breathing and therefore the image artifacts on a two-beat time scale.

For black blood and bright blood MRI acquisition, a gadolinium based contrast product is advantageously injected intravenously into the patient, 10 to 15 minutes before the application of the acquisition sequences so as to obtain images exhibiting maximum contrast between scars and healthy tissues and blood. At the heart, the contrast is rapidly eliminated from healthy myocardium, which is low in interstitial tissue, but accumulates prolongedly in myocardial scars. The effect of gadolinium is to shorten the relaxation time T1 of the tissues where it accumulates. The relaxation of the magnetization of scars following a magnetization pulse is thus faster than that of blood and healthy myocardium.

In the first instance, the aim is to generate images exhibiting black blood contrast. In an image of this type, the intensity of the pixels corresponding to blood and healthy muscle is null (black pixels) or substantially null because it is acquired when the longitudinal magnetization of the blood is null.

In order to generate such an image IM₁, the device DISPO_RF generates an inversion-recovery acquisition sequence.

This acquisition sequence comprises a 180° inversion pulse noted Rfi in FIG. 2, which shifts the longitudinal magnetization of the tissues of the imaged area in the opposite direction, i.e. which inverses the longitudinal magnetization. FIG. 2 shows that the magnetization of the area to be imaged changes from Mz to −Mz under the effect of the inversion pulse Rfi. Due to longitudinal relaxation, the longitudinal magnetization of the different tissues present in the area to be imaged increases to return to its initial value, passing through the null value. Naturally, the return rates of the magnetizations of the different tissues are different.

In a manner known per se, the preparation sequence next comprises a preparation module, for example an adiabatic T1rho preparation, noted T1rho or TAprep in FIG. 2 of TSL (denoting “Time of Spin Lock”) duration.

Other preparations, such as a T2prep, a magnetization transfer preparation MT may be used.

It is noted in the present application that the preparation may sometimes be defined as the T1rho preparation or sometimes be defined as the T1rho preparation preceded by the 180° Rfi inversion sequence. The second image IM2 being acquired without 180° inversion pulse, but only with a T1rho preparation, the preparation in this case is limited to the T1rho preparation.

The preparation sequence) (Rfi (180°, T1rho) is configured such that the longitudinal magnetization of blood and that of healthy myocardium are nulled at the same instant TI, see the position of the NP point of the A(blood) and A(Musc) curves. The inversion time TI corresponds to a time measurement between the transmission of the Rf signal at 180 and the acquisition module ACQ1. The instant TI corresponds to the duration noted D₁ in the present application. Indeed, the invention also applies to cases in which the measurement of the magnetization would not necessarily be calculated at the inversion time, but possibly at another moment depending on the desired result.

Regardless of whether an acquisition window or a read window is noted for each beat, they are noted ACQ₁ for the read windows relating to black blood acquisition and ACQ₂ for the read windows relating to bright blood acquisition.

It is noted that in FIG. 2, at this same instant TI, the longitudinal magnetization of the scars A(Cica) is significantly greater than zero, indeed the PP point is above zero. By acquiring the signals from the area to be imaged at this instant TI, an image is obtained exhibiting a very high contrast between the pixels corresponding to blood and healthy myocardium, which are black, and scars, which are generally bright.

The inversion-recovery sequence then comprises a read sequence ACQ₁ comprising a 90° pulse and a read gradient to read the transverse magnetization of the area to be imaged.

The inversion time TI is the duration separating the 180° pulse and the acquisition window ACQ₁. The time marker of the acquisition window ACQ₁ is imposed upstream in the ECG, generally in diastole. It is chosen as the instant when the muscle and blood signals cancel out, i.e. where the longitudinal magnetizations of blood and myocardium cancel out in order to generate the image exhibiting the best contrast. In order to obtain the best contrast between myocardial scars and blood and healthy myocardium, it is sought to initiate the inversion Rf signal at an earlier instant to cause the cancelation of the signals at a duration TI.

The sequence is followed by a bright blood acquisition phase performed at the beat following the black blood acquisition. In this phase, a second image IM₂ is acquired. The preparation phase is noted TBprep in FIG. 2. According to one embodiment, the preparation TBprep could be different from that of the preparation TAprep used to black blood image the area of the heart, however it is preferable that the black blood preparations TAprep and bright blood preparations TBprep are identical. The invention therefore relates to other preparations, however, in the remainder of the description, the bright blood acquisition phase is described with the same bright blood preparation as the black blood preparation, for example a T1rho preparation.

The duration D₂ separating the time marker from the acquisition window AQ₂, which is set for each beat, and the time marker corresponding to the bright blood preparation TBprep may be adjusted to optimize the bright blood contrast in order to best image muscle contours relative to blood.

The measurement of magnetization gradients is configured for an acquisition ACQ₂ of the bright blood image at the same inter-beat marker as the black blood image. Thus, in FIG. 2, the read phase ACQ₂ is located at a duration of the previous QRS complex identical to the duration between the QRS of the beat preceding the read phase ACQ₁.

When reading magnetizations at this same instant, it is possible to obtain an image in which the pixels of blood areas are represented in white, i.e. with high luminance, and in which the pixels of myocardial tissues are slightly less luminous than those of blood. Thus, it is possible to obtain the 2D topology of the section allowing the visualization of blood and myocardium areas in relation to each other.

It is understood that by combining the images IM₁ and IM₂ it will be possible to locate and detect the scar if it is present.

To this end, the invention makes it possible to perform successive measurements of the sequencing of these two black blood IM₁ and bright blood IM₂ images on a plurality of sections of the heart to study the entire heart and by taking into account a plurality of images per section in order to make the image analysis more robust.

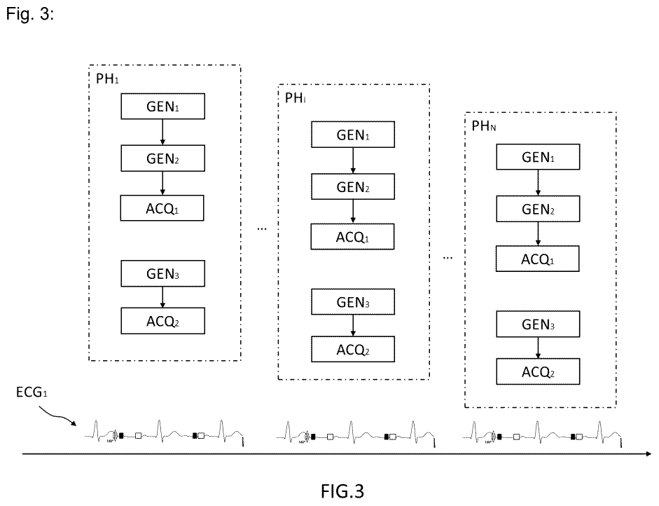

FIG. 3 shows an example of sequencing of these phases noted Phi, with i=1 to N, over a plurality of consecutive beat pairs. The phases PHi comprise each time on two beats:
First beat: a generation GEN₁ of an inversion RF signal, a preparation phase generated and noted GEN₂ and a read phase noted ACQ₁;
Second beat: the generation of a GEN₃ preparation phase and a read phase noted ACQ₂.

FIG. 4 shows a 3D view of a heart including a plurality of K section planes noted PCₖ, with k=1 to K. FIG. 4 makes it possible to represent a plurality of section planes each comprising a plurality of images acquired thanks to the method of the invention. The images of the section plane PCₖ are shown, the others are not shown in FIG. 4. The images are noted respectively IM₁^{k}(i) for the i^{th} image acquired of the section PCₖ, with i=1 to N.

According to one embodiment, a first acquisition of the image of the heart makes it possible to evaluate the number of section planes and therefore the number of acquisition repetitions. If the heart is considered small size, between 7 and 9 section planes may be defined, if the heart is larger, between 14 and 20 sections may be defined. If the heart is medium size, the number of sections may be defined between 10 and 13 sections.

This estimation may be made at the beginning of the examination, for example during a pre-sequence.

According to another example, the evaluation of the heart size is determined automatically during the first acquisitions of the method of the invention, during the first beats. This size can automatically allow a dynamic calculation to be performed in such a way as to acquire images according to a given number of sections.

According to an example of a complete sequence of 3 min with a pulse of 60 beats per min, considering a configuration of 15 section planes for the organ, we would have a total of 3×60=180 images, of which 90 black blood images and 90 bright blood images. Such a configuration made it possible to acquire 6 images {IM₁^{k}(1), IM₁^{k}(2), IM₁^{k}(3), IM₁^{k}(4), IM₁^{k}(5), IM₁^{k}(6)} per black blood section plane and 6 acquired images {IM₂^{k}(1), IM₂^{k}(2), IM₂^{k}(3), IM₂^{k}(4), IM₂^{k}(5), IM₂^{k}(6)} per black blood section plane.

One advantage of the method of the invention is to be carried out in free breathing. For this purpose, it is necessary that the images acquired in a same section are processed in order that they are not affected by the effects of free breathing. The breathing rhythm is a priori different from the heart rhythm. Even if correlations exist between these two rhythms, it is possible that breathing will accelerate while the heartbeat remains stable or vice versa.

The problem is that during the second read ACQ₂, the breathing was able to significantly change the position of the heart, which has the consequence that the images acquired at another beat of the same section may be offset with respect to an image acquired previously in the same section.

According to one embodiment, the images of each section resulting from the black blood acquisition are registered from a non-rigid image registration algorithm. The bright blood images acquired for each section are registered identically to the chosen black blood method. It is possible to choose a different algorithm for processing black blood and bright blood images, but it is preferable to choose the same algorithm for ease of implementation. The registration significantly improves the image that will be merged from a plurality of images acquired in a same section, because the contrast is better and breathing artifacts have been able to be compensated for thanks to the non-rigid algorithm.

According to a first example, a non-rigid algorithm is an algorithm based on the method of mutual information between images based on statistical relationships. The function to be optimized may be implemented by a statistical similarity criterion. One benefit of this method is that the pairing between homologous attributes of images of a same section is independent of their geometric position.

According to a second example, a non-rigid algorithm based on a transformation model is implemented. The transformation model makes it possible to determine functions enabling the difference between two images to be minimized. The deviation may be translated into a geometric error to be minimized. Different approaches may be used such as those based on extracting from each of the images geometric primitives or shape descriptors such as protruding points, shape singularities or contours. A parametric or non-parametric approach may be used.

According to one example of optimization of a transformation model or a similarity criterion, the least squares method may be used.

Other optimization methods may be implemented such as gradient descent. However, the latter method applies more particularly to image intensities and is not optimal in the context of the invention since it is sought to optimize the sharpness and contrast of the merged image. Nonetheless, the invention includes this embodiment.

The registration may be performed by choosing a reference image and determining a transformation function of the other images of the same section in relation to that image. Each image is then registered by optimizing a transformation to obtain the reference image according to a geometric criterion from the image considered.

When a plurality of images are registered together in a section plane, it is possible to perform operations aimed at merging these images in order to produce a single merged image per section plane.

According to one embodiment, a step of averaging the images of a same section is performed so as to reduce noise and increase the signal to noise ratio.

Averaging has the advantage of conserving the detail of the image, since it increases the signal to noise ratio SNR. This technique makes it possible to smooth out noise to reduce residual image artifacts. Further, averaging makes it possible to improve the bit depth of the digital image beyond what is possible with a single image.

One benefit of the step of averaging images taken from the same section is to reduce the maximum deviation. The amplitude of the noise decreases as the square root of the number of images used, i.e. with only 4 images, the amplitude of the noise can be reduced by a factor of two. According to an example of an acquisition in free breathing of a duration of 2 min, it is possible to collect 4 to 5 images per section plane, which makes it possible to obtain good noise reduction performances.

Each set of images acquired on the one hand in black blood and on the other hand in bright blood are respectively merged so as to produce a single black blood image IM_{1F}^{k} and a single bright blood image IM_{2F}^{k} per section plane PCₖ.

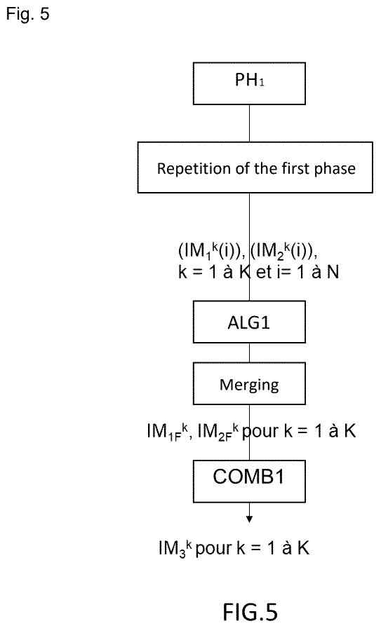

Put another way, a single merged black blood image IM_{1F}^{k} and a merged bright blood image IM_{2F}^{k} are produced per section plane PCₖ as seen in FIG. 5.

According to one embodiment, the method of the invention then comprises a step of coloring the most luminous pixels of the black blood image. The most luminous pixels correspond in particular to scar given that the signals from muscle and blood are nulled and are represented by pixels of low intensity, i.e. black. The colorization of the scar makes it possible to identify and represent the scar contour.

One benefit of colorization is to allow the scar to be clearly identified and to use this representation combined with the bright blood image in which the contours of the muscles are represented. Thus, such a combination makes it possible to identify and locate the scar notably with respect to muscle and blood near the scar.

The combination COMB1 of the black blood IM_{1F}^{k} and bright blood IM_{2F}^{k} images may be performed for each section plane PCₖ. Thus, the same combination operation makes it possible to end up with a plurality of combined black blood—bright blood images to produce a single image IM₃^{k} per section plane PCₖ.

The combination COMB1 of the images may be performed in different ways. According to a first example, the images are overlaid on each other. The overlays may comprise taking into account a variation in the opacity of the black blood image in order to properly visualize the contours of the bright blood image. The opacity of black blood images may be configured to have a value comprised between 40% and 90%, for example 75%. According to a second example, the pixels of the scar that have a luminance greater than a given threshold are extracted to be integrated into the bright blood image.

Other image combination methods may be used in order to produce a single image from the black blood image and the bright blood image per section.

In other words, for each section plane PCₖ, a single combined image IM₃^{k} is produced from the merged black blood image IM_{1F}^{k} and the merged bright blood image IM_{2F}^{k}.

One benefit of the method of the invention is to generate a plurality of reconstructed images per section plane from a black blood image and a bright blood image. Depending on the size of the heart, between 8 and 20 section planes may be selected. The method of the invention makes it possible to display a reconstructed image for each section plane to a user, more particularly a radiologist. Each image allows the presence or absence of a scar to be displayed by locating it accurately in relation to the anatomy of the heart, the myocardium, and the blood volumes surrounding the imaged area.

1. A method for reconstructing an image of a patient's heart from a magnetic resonance imaging device generating a magnetic field comprising:
a first phase of image generation including:
acquiring electrical activity of the patient's heart;
generating a first 180° inversion radio frequency signal to inverse a longitudinal magnetization of tissues of an imaged area, said first inversion radio frequency signal being generated between two QRS complexes of the acquired electrical activity, so-called first inter-beat phase;
generating a first magnetization preparation including a set of pulses following the generation of the first inversion radio frequency signal in the same first inter-beat phase;
acquiring a first 2D image by a magnetization measurement of the imaged area, said acquiring being performed after a first predefined duration following the generation of the first magnetization preparation, said acquiring being synchronized with the heart rhythm at a first time marker of the same inter-beat phase;
generating a second magnetization preparation including a set of pulses in a second inter-beat phase following the first inter-beat phase;
acquiring a second 2D image by a magnetization measurement of the imaged area, said acquiring being performed after a second predefined duration following the generation of the second magnetization preparation, said acquiring being synchronized with the acquisition of a heart rhythm at the same first time marker of the second inter-beat phase as the first marker of the first inter-beat phase;
repeating the first phase, to generate a subset of first images and a subset of second images of each section plane of a plurality of section planes of the heart, in inter-beat phases following the first and second inter-beat phases;
applying a non-rigid registration algorithm to each subset of first images) of one of the section planes to register said first images) of the subset of first images together;
applying a non-rigid registration algorithm to each subset of second images of one of the section planes to register said second images of the subset of second images together;
merging, for each of the section planes, on the one hand the first images registered together to produce a first merged image and on the other hand the second images registered together to produce a second merged image;
generating, for said section planes, of a set of third 2D images, each third image corresponding to a first combination made between the first merged image and the second merged image produced for one of the section planes.

2. The method according to claim 1, wherein the first duration being determined such that the first acquired images are images having a first contrast making it possible to display black blood images and the second duration being determined such that the second acquired images are images having a second contrast making it possible to display bright blood images.

3. The method according to claim 1, wherein the registration step is followed by the steps of:
averaging the first registered images to produce a first merged image for each section plane;
averaging the second registered images to produce a first merged image for each section plane.

4. The method according to claim 1, wherein the first duration is defined in such a way that the longitudinal magnetization of blood and that of healthy myocardium are nulled at the same instant TI of the acquisition of the first 2D image.

5. The method according to claim 1, wherein the determination of the first duration is automatically calculated from a computer-implemented method of an optimal inversion time performed from a processing of at least one image acquired from an MRI pre-sequence.

6. The method according to claim 1, comprising a step of coloring the pixels of each first merged image having a luminance greater than a given threshold, the coloring step being performed prior to the first combination.

7. The method according to claim 1, wherein the first combination is an overlay of the first image on the second merged image.

8. The method according to claim 1, wherein:
each generation of a new first image and a new second image in a new heart section plane is performed after:
all the repetitions of the first phase to generate a plurality of first images }_{i[1-N]}) and second images }_{i[1-N]}) of a preceding section plane of the heart.

9. The method according to claim 1, comprising:
a display of displaying a set of first merged images for each section plane;
a display of displaying a set of second merged images for each section plane;
a display of displaying a set of third 2D images for each section plane;
each first image of a given section plane being displayed in the immediate vicinity of the second image and the third image of the same given section plane.

10. The method according to claim 1, wherein the first merged image for each section plane corresponds to a black blood image of a section of the heart, the second merged image for each section plane corresponds to a bright blood image of a section of the heart, the third image for each section plane corresponding to an image of a section of the organ on which a scar, if present, is displayed in color.

11. The method according to claim 1, wherein the first and second magnetization preparation are T1rho weightings.

12. The method according to claim 1, wherein the first magnetization preparation and/or the second magnetization preparation is a preparation of the T2prep type or a magnetization transfer preparation.

13. The method according to claim 1, wherein the generation of a second magnetization preparation including a set of pulses in an inter-beat phase is followed by a step of filtering the acquired images corresponding to fatty areas of the imaged organ.

14. Th method according to claim 1, wherein the first images and the second images are acquired successively in a manner synchronized with the electrical activity of the heart between two QRS complexes over a plurality of minutes in free breathing during a single examination.

15. The method according to claim 1, wherein the first images and the second images are acquired on a set of 8 to 20 section planes of the organ, the phase of reiteration of the acquisitions of a plurality of first images }_{i[1-N]}) and second images }_{i[1-N]}) in the same section plane making it possible to acquire between 2 and 10 images of first images }_{i[1-N]}) and between 2 and 10 images of second images }_{i[1-N]}) per section plane.

16. A magnetic resonance imaging system comprising a magnetic field generator and a radio frequency device, an electrocardiograma and a processing device, a display for displaying the generated images, said system being configured to implement the method of claim 1.
